(19) United States
(12) Patent Application Publication
(10) Pub. No.: US 2008/0113387 A1
(43) Pub. Date: May 15, 2008

(54) Immunoassay for Prion Disease

(75) Inventors: Raymond Bujdoso, Cambridge (GB); Alana Thackray, Cambridge (GB)

Correspondence Address: WOLF GREENFIELD & SACKS, P.C., 600 ATLANTIC AVENUE, BOSTON, MA, 02210-2206, US

(73) Assignee: Cambridge Enterprise Limited, Cambridgeshire (GB)

(21) Appl. No.: 11/883,004

(22) PCT Filed: Jan. 25, 2006
(86) PCT No.: PCT/GB06/00252

§ 371 Date: Jul. 25, 2007

(30) Foreign Application Priority Data
Jan. 25, 2005 (GB) 0501564.9

(51) Int. Cl. G01N 33/567; G01N 33/53; C12N 5/06

(52) U.S. Cl. 435/7.2; 435/326; 530/388.26

(57) ABSTRACT

This invention relates to the detection or assessment of prion disease in an individual by determining the binding of monoclonal antibodies specific for PrP to the surface of blood cells obtained from the individual. The binding of such antibodies is shown to correlate with the progression of prion disease. Methods of detecting or assessing prion disease are provided, along with kits and reagents.

This invention relates to methods of testing for the presence of prion disease in an individual, in particular blood-based methods that can be performed ante-mortem.

Prion diseases, such as scrapie in sheep, bovine spongiform encephalopathy (BSE) in cattle and Creutzfeldt-Jakob Disease (CJD) in humans, are transmissible chronic neurodegenerative disorders characterized by the accumulation of PrP^{sc} [the abnormal disease-specific conformation of prion-related protein (PrP)], an abnormal isomer of the host protein PrP^{c} (normal cellular PrP) (Prusiner, S. B. (1982) Science 216, 136-44).

During the pre-clinical phase of prion disease induced by peripheral inoculation, prion infectivity and PrP^{sc} can be detected within peripheral lymphoid tissue. At these sites, PrP^{sc} depositions may be found in tingible body macrophages (TBMs) and follicular dendritic cells (FDCs), although replication of the infectious prion agent appears to be sustained by FDCs (McBride, P. A. et al. (1992) J. Pathol. 168, 413-8; Aguzzi, A. (2003) Adv. Immunol. 81, 123-71). The temporal appearance of PrP^{sc} in the lymphoreticular system, the peripheral nervous system (PNS) and eventually the central nervous system (CNS) of prion infected individuals is indicative of spread by the nervous system (Beekes, M. and McBride, P. A. (2000) Neurosci. Lett. 278, 181-4; van Keulen, L. J. et al (2000) Arch. Virol. Suppl. 57-71). This is supported by extensive studies in transgenic and gene-knock-out mice, which demonstrate that neuroinvasion occurs principally via the PNS (Glatzel, M. et al. (2001) Neuron 31, 25-34; Lasmezas, C. I. et al (1996) J. Virol. 70, 1292-5; Race, R. et al. (2000) J. Virol. 74 828-33; Baldauf, E. et al (1997) J. Gen. Virol. 78 1187-97).

It has been shown that prion disease can be transmitted through transfusion of whole blood, or buffy coat, from natural scrapie-infected, or BSE-experimentally infected sheep, into recipient sheep (Hunter, N. et al (2002) J. Gen. Virol. 83, 2897-905; Houston, F. et al (2000) Lancet 356, 999-1000). Similarly, prion infectivity has been detected in whole blood and in buffy coat from mice infected with a human-derived strain of variant CJD during both the pre-clinical and clinical phases of disease (Cervenakova, L. et al (2003) Transfusion 43, 1687-94). The presence of detectable infectivity in blood of experimentally infected animals has reinforced concerns that human blood supplies may be contaminated with prion infectivity (Llewelyn, C. A. et al (2004) Lancet 363, 417-21).

Polymorphisms in the ovine PrP protein at amino acid residues 136, 154 and 171 are associated with variation in susceptibility to natural scrapie. V136R154Q171 (VRQ) or A136R154Q171 (ARQ) animals show susceptibility to scrapie, whilst those that express A136R154R171 (ARR) show resistance (Clouscard, C. et al (1995) J. Gen. Virol. 76, 2097-101; Goldmann, W. et al (1994) J. Gen. Virol. 75, 989-95).

Circulating peripheral blood mononuclear cells (PBMCs) of sheep (Herrmann, L. M. et al (2001) Haematologica 86, 146-5; Holada, K. et al(2002) Blood 100, 341-3) and other species (Holada, K. and Vostal, J. G. (2000) Br. J. Haematol. 110 472-80), express PrP^{c} on their cell surface. It has been shown that PrP^{c} expressed on the surface of ovine PBMCs displays conformational variation between scrapie-susceptible and -resistant genotypes, and between different susceptible allelic variants (Thackray, A. M. et al. (2004) Biochem. J. 381, 221-9). This indicates that ovine PrP expressed on the surface of blood cells is capable of structural flexibility.

The present inventors have recognized that the binding of monoclonal antibodies specific for PrP to the surface of blood cells correlates with the progression of experimental prion infection. These antibodies may therefore be useful in diagnostic tests for prion diseases.

An aspect of the invention provides a method of detecting prion disease in an individual or assessing the progression of prion disease in an individual comprising;
contacting blood cells obtained from said individual with an antibody which specifically binds to PrP, and
determining the binding of said antibody to said cells.

A change, for example an increase or a decrease, in the binding of said antibody to blood cells from the individual, relative to controls, may be indicative of the presence of prion disease in the individual or may be indicative of the progression of prion disease in the individual (i.e. the stage of the prion disease in an individual). Suitable controls include blood cells from a healthy individual (i.e. an individual not suffering from a prion disease). The healthy individual may be age-matched with the individual being tested.

In some embodiments, an increase in the binding of antibody relative to controls is indicative of the presence or progression of prion disease in the individual. Binding may increase as the disease progresses, so the extent of binding may be indicative of the status of the disease. For example, high levels of binding relative to controls may be indicative of advanced prion disease.

Suitable antibodies for use in such embodiments include antibodies which specifically bind to residues 25 to 88 of ovine PrP or the corresponding residues in PrP^{c} from other sources. Examples of such antibodies include T188, T164 and T325, which are described elsewhere herein. Other suitable antibodies include antibodies which specifically bind to residues 89 to 233 of ovine PrP, in particular residues 217-232 of ovine PrP. Suitable antibodies include V26, which specifically binds to the epitope CITQYQRESQAYYQRG.

In some preferred embodiments, an antibody which specifically binds to the epitope CITQYQRESQAYYQRG, such as V26, may be employed in a method described herein. A method of detecting prion disease in an individual or assessing progression of a prion disease in an individual may comprise;
contacting a sample of blood cells obtained from said individual with an antibody which specifically binds to the epitope CITQYQRESQAYYQRG, and
determining the binding of said antibody to said cells.

An antibody which specifically binds to the epitope CITQYQRESQAYYQRG may specifically bind to residues 217-232 of ovine PrP or the corresponding residues of other mammalian PrP's, such as bovine, human or cervid PrP.

In other embodiments, a decrease in the binding of antibody relative to controls is indicative of the presence or progression of prion disease in the individual. The extent of the decrease in binding may be indicative of the progression of the prion disease. For example, low levels of binding relative to controls may be indicative of advanced prion disease. Suitable antibodies may specifically bind to an epitope comprising or influenced by residue 171 of ovine PrP and include antibody V468, which is described elsewhere herein.

The antibodies T188, T164, T325, V26 and V468 and their production are described in more detail in Thackray et al Biochem. J. (2004) 381, 221-229

The blood cell sample may be comprised in a blood sample obtained from the individual or may be a population or sample of blood cells isolated and/or purified from a blood sample obtained from the individual. Suitable blood cells include peripheral blood mononuclear cells (PBMCs).

Blood cells may be isolated and/or purified from a blood sample obtained from the individual using conventional techniques; for example by venepuncture and, optionally, density gradient centrifugation

An antibody which specifically binds to a target epitope may show significant binding to the target epitope when the epitope is present and accessible in a polypeptide, for example a PrP^{c} protein, and may show little or no binding to peptides or polypeptides in which the target epitope is absent or inaccessible to antibody. In particular, an antibody may show no significant binding to other proteins present in the sample or to other surface components of mammalian blood cells. For example, an antibody which specifically binds to the epitope CITQYQRESQAYYQRG may show no significant binding to the ovine PrP^{c} protein outside residues 217-232, to other mammalian PrP^{c} proteins outside the corresponding target region of those proteins, or to other surface components of mammalian blood cells.

Generally, an antibody which specifically binds to a target epitope may have a binding affinity greater than about 10⁵ moles/liter (e.g., 10⁶, 10⁷, 10⁸, 10⁹, 10¹⁰, 10¹¹, and 10¹² or more moles/liter).

An antibody suitable for use in the present methods may be monoclonal or polyclonal and may be produced using conventional techniques. For example, an antibody may be produced by immunising a mammal (e.g. mouse, rat, rabbit, horse, goat, sheep or monkey) with a PrP antigen, for example a peptide comprising or consisting of the epitope CITQYQRESQAYYQRG. Antibodies may be obtained from the immunised animals using any of a variety of techniques known in the art, and screened, preferably using binding of antibody to the PrP^{c} antigen. For instance, Western blotting techniques or immunoprecipitation may be used (Armitage et al. (1992) Nature 357: 80-82). Isolation of antibodies and/or antibody-producing cells from an animal may be accompanied by a step of sacrificing the animal.

Monoclonal antibodies may be produced by isolating antibody producing cells from the immunised mammal, fusing them with immortalised cells to produce a population of antibody producing hybridoma cells. The population may then be screened to identify a hybridoma cell that produces an antibody which displays optimal binding characteristics.

Methods of producing hybridoma cells and monoclonal antibodies are well known in the art (see, for example, Harlow et al Antibodies: A Laboratory Manual, Cold Spring Harbor Laboratory (Cold Spring Harbor, N.Y., 1988) pp. 353-355) and are described in more detail below.

As an alternative or supplement to immunising a mammal with a peptide, an antibody specific for an epitope may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with a peptide comprising the epitope, or may be one constructed using sequences obtained from an organism which has been exposed to the antigen of interest.

The binding of antibodies to blood cells may be determined in the methods described herein by any appropriate means or assay format. Tagging with individual reporter molecules is one possibility. For example, the antibody, or a second antibody which binds to it, may be tagged with a reporter molecule. The reporter molecule may directly or indirectly generate detectable, preferably measurable, signal.

Where required, linkage of reporter molecules may be direct or indirect, covalent, e.g. via a peptide bond, or non-covalent. Linkage via a peptide bond may be as a result of recombinant expression of a gene fusion encoding binding molecule (e.g. antibody) and reporter molecule. Linkage via a non-covalent bond may be a result of a binding between a biotinylated antibody and a streptavidin/avidin linked reporter molecule.

Reporters include fluorochromes such as fluorescein, rhodamine, phycoerythrin and Texas Red, chromogenic dyes such as diaminobenzidine, macromolecular colloidal particles or particulate material such as latex beads that are coloured, magnetic or paramagnetic, and biologically or chemically active agents that can directly or indirectly cause detectable signals to be visually observed, electronically detected or otherwise recorded.

Biologically or chemically active agents include enzymes, which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed. Further examples include horseradish peroxidase and chemiluminescence.

The signals generated by individual antibody-reporter conjugates may be used to derive quantifiable absolute or relative data of the relevant antibody binding in samples (normal and test) and, in particular may be used to determine the proportion of cells in the sample which are bound by the antibody. Any change, for example an increase or decrease, in the proportion of cells in the sample which bind to the antibody relative to the proportion of cells from a control sample which bind to the antibody may be indicative of prion disease in said individual, as described herein.

Any convenient assay format may be employed to carry out the present methods. For example, fluorescence activated cell sorting (FACS) or flow cytometry may be employed or an immunoassay such as ELISA. The binding of anti-PrP antibodies to cells may be indicated by a fluorescent reporter. The reporter may, for example, be covalently or non-covalently bound to the anti-PrP antibody or to a second antibody which binds to the anti-PrP antibody. Cells labelled with the fluorescent reporter may then be identified and counted using a FACS apparatus [e.g. FACSCalibur® (Becton Dickinson, Mount View, Calif., U.S.A.)]. Suitable fluorescence activated cell sorting techniques are well known in the art.

The mode of determining binding is not a feature of the present invention and those skilled in the art are able to choose a suitable mode according to their preference and general knowledge.

The methods herein may be used to detect a prion disease selected from the group consisting of Creutzfeldt-Jakob disease (CJD), Kuru, Gerstmann-Sträussler-Scheinker syndrome, scrapie, chronic wasting disease and BSE. CJD may include sporadic, familial, iatrogenic and variant CJD. For example, the methods may be used to detect Creutzfeldt-Jakob disease (CJD), Kuru and Gerstmann-Straussler-Scheinker syndrome in humans, scrapie in sheep and goats, chronic wasting disease in elk and deer (cervids) and/or BSE in cattle.

In some embodiments, the individual may display no behavioural changes or clinical symptoms associated with prion disease. In other embodiments, the individual may display one or more behavioural changes or clinical symptoms associated with prion disease.

Another aspect of the invention provides an isolated anti-PrP antibody that specifically binds to the epitope CITQYQRESQAYYQRG.

The antibody may, for example, specifically bind residues 217-232 of ovine PrP, or the corresponding region in other mammalian PrP proteins, in particular bovine, human or cervid PrP.

The antibody may be labelled with a detectable reporter, for example a fluorescent reporter, or a tag for binding a detectable reporter as described above.

The antibody may be polyclonal or monoclonal. Methods of producing such antibodies are well known in the art and described elsewhere herein.

Another aspect of the invention provides a hybridoma cell which produces an anti-PrP antibody that specifically binds to the epitope CITQYQRESQAYYQRG as described above. Techniques for the production of hybridoma cells are well known in the art. For example, a hybridoma may be produced by a method comprising;
immunising a non-human mammal with a peptide comprising or consisting of the sequence CITQYQRESQAYYQRG,
producing one or more fusions of antibody producing cells from said mammal and immortalised cells to provide a population of hybridoma cells and;
screening said population to identify a hybridoma cell which produces an antibody which binds to said peptide.

The population of hybridoma cells is preferably screened by testing the binding of antibodies produced by cells of the population to the peptide sequence CITQYQRESQAYYQRG. For instance, Western blotting techniques or immunoprecipitation may be used (Armitage et al. (1992) Nature 357: 80-82).

Hybridoma cells identified as producing antibodies with desirable binding properties may be isolated and/or purified from the population. Following isolation, the hybridoma may be expanded, maintained and/or cultured in a culture medium using methods which are well-known in the art.

Antibody molecules which specifically bind the target peptide may be isolated and/or purified from said culture medium, for example for use in accordance with the present methods. A method of producing an anti-PrP antibody may comprise;
culturing a hybridoma cell as described above in a culture medium; and,
isolating said antibody from said medium.

Other aspects of the invention provide the use of a peptide consisting of the sequence CITQYQRESQAYYQRG in the production of antibodies for use in detecting prion infection and the use of an antibody which specifically binds to the sequence CITQYQRESQAYYQRG in the detection of prion infection.

An anti-PrP antibody as described herein may form part of a kit for detecting or diagnosing a prion disorder e.g. in a suitable container such as a vial in which the contents are protected from the external environment.

A kit for detecting a prion disorder may comprise:
an anti-PrP antibody that specifically binds to the epitope CITQYQRESQAYYQRG as described above, and;
detection reagents for determining binding of the antibody to blood cells.

The detection reagents may comprise a second antibody which binds to the anti-PrP antibody.

The detection reagents may comprise a detectable reporter, for example a fluorescent reporter. The anti-PrP antibody or the second antibody may be labelled with the detectable reporter or may be labelled with a tag for binding a detectable reporter. Suitable tags include biotin.

The detection reagents may further comprise a substrate, for example a chromogenic, fluorescent or chemiluminescent substrate, which reacts with the reporter to produce a signal, buffer solutions etc.

A kit may further comprise one or more articles and/or reagents for performance of the method, such as means for providing the test sample itself, e.g. a syringe for removing a blood sample (such components generally being sterile), means for handling and/or storing the sample, and means for isolating and/or purifying blood cells, in particular PBMCs, from said sample.

The kit may also include instructions for use of the antibody, e.g. in a method of detecting a prion disorder in a test sample, as described herein.

Various further aspects and embodiments of the present invention will be apparent to those skilled in the art in view of the present disclosure. All documents mentioned in this specification are incorporated herein by reference in their entirety.

The invention encompasses each and every combination and sub-combination of the features that are described above.

Certain aspects and embodiments of the invention will now be illustrated by way of example and with reference to the figures described above and tables described below.

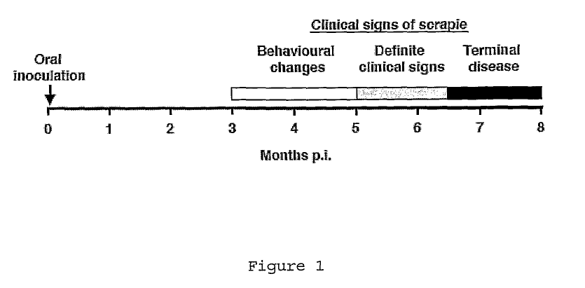

FIG. 1 shows a diagram of the appearance of clinical signs in scrapie inoculated VRQ/VRQ lambs.

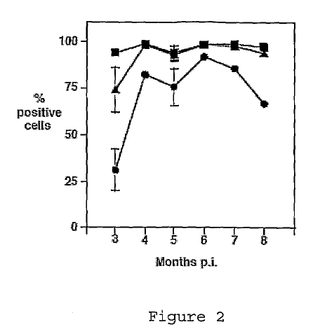

FIG. 2 shows the percentage of PBMCs from scrapie-infected sheep reactive with N-terminal-specific anti-PrP monoclonal antibodies. Ovine PBMCs from VRQ/VRQ sheep were collected at various time points following oral inoculation with scrapie brain material and analyzed by flow cytometry with anti-PrP monoclonal antibodies T164 (circles), T188 (triangles) or T325 (squares) as described in the Materials and Methods. n=6 for months 3-5 p.i.; n=3 for months 6-7 p.i.; and n=2 for month 8 p.i. Results are shown as mean±S.D.

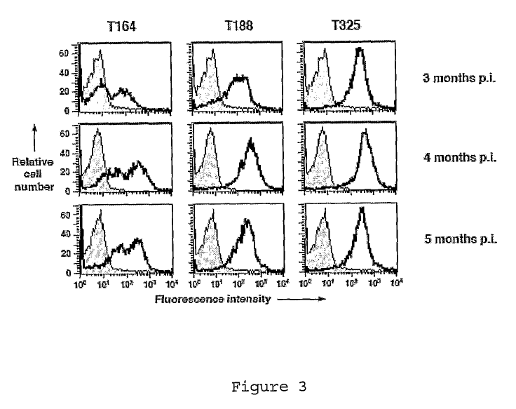

FIG. 3 shows the reactivity of N-terminal-specific anti-PrP monoclonal antibodies with PBMCs from scrapie-infected sheep. Representative flow cytometry profiles of ovine PBMCs collected at 3-5 months p.i. with scrapie material following reactivity with either T164, T188 or T325. Profiles shown are representative of 6 out of 6 sheep for each anti-PrP monoclonal antibody. Shaded peak represents control fluorescence; black line represents T164, T188 or T325 fluorescence.

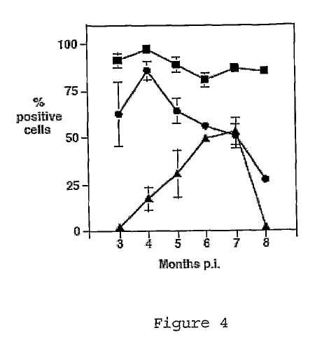

FIG. 4 shows the percentage of PBMCs from scrapie-infected sheep reactive with C-terminal-specific anti-PrP monoclonal antibodies. Ovine PBMCs from VRQ/VRQ sheep were collected at various time points following oral inoculation with scrapie brain material and analyzed by flow cytometry with anti-PrP monoclonal antibodies V468 (circles), V26 (triangles) or A516 (squares) as described in the Materials and Methods. n=6 for months 3-5 p.i.; n=3 for months 6-7 p.i.; and n=2 for month 8 p.i. Results are shown as mean±S.D.

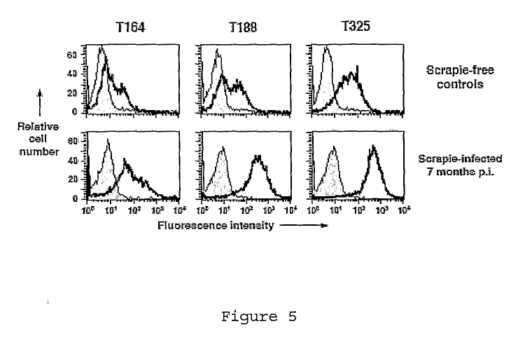

FIG. 5 shows the reactivity of N-terminal-specific anti-PrP monoclonal antibodies with PBMCs from terminal scrapie-infected sheep and age-matched scrapie-free controls. Representative T164, T188 or T325 flow cytometry profiles of ovine PBMCs collected at either 7 months p.i. with scrapie material, or from age-matched scrapie-free sheep are shown. Shaded peak represents control fluorescence; black line represents T164, T188 or T325 fluorescence. n=3 for scrapie-infected sheep; n=4 for age-matched scrapie-free controls.

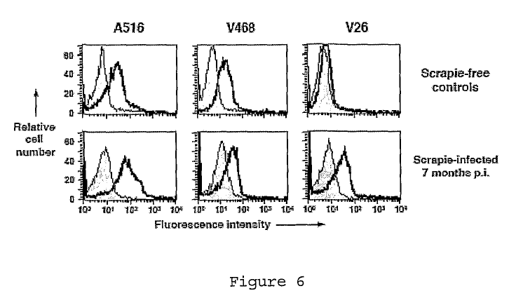

FIG. 6 shows the reactivity of C-terminal-specific anti-PrP monoclonal antibodies with PBMCs from terminal scrapie-infected sheep and age-matched scrapie-free controls. Representative A516, V468 or V26 flow cytometry profiles of ovine PBMCs collected at either 7 months p.i. with scrapie material, or from age-matched scrapie-free sheep are shown. Shaded peak represents control fluorescence; black line represents A516, V468 or V26 fluorescence. n=3 for scrapie-infected sheep; n=4 for age-matched scrapie-free controls.

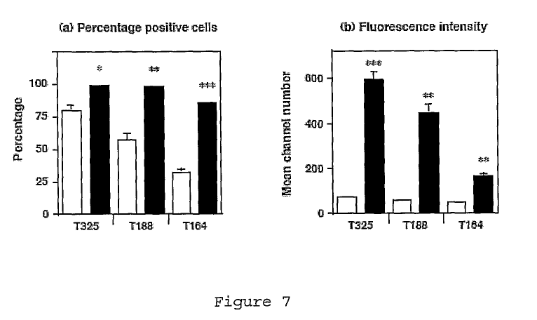

FIG. 7 shows a quantitation of N-terminal PrP flow cytometric analysis (a) Percentage positive cells (mean±SD) and (b) mean channel number (mean±SD) are shown for PBMCs from scrapie-infected sheep at 7 months p.i. (black bar) (n=3) and scrapie-free sheep (white bar) (n=4) stained with T325, T188 or T164. Statistical analysis of the data in FIG. 7(a): *p<0.05 (comparison with scrapie-free equivalent). **p<0.01 (comparison with scrapie-free equivalent). ***p<0.001 (comparison with scrapie-free equivalent).

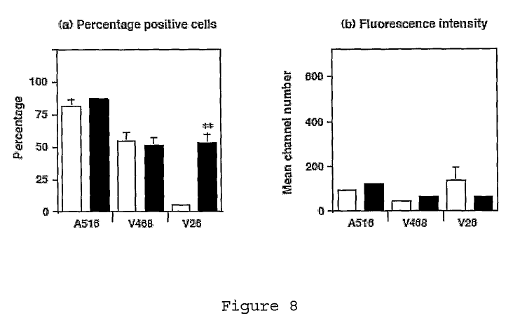

FIG. 8 shows a quantitation of C-terminal PrP flow cytometric analysis. (a) Percentage positive cells (mean±SD) and (b) mean channel number (mean±SD) are shown for PBMCs from scrapie-infected sheep at 7 months p.i. (black bar) (n=3) and scrapie-free sheep (white bar) (n=4) stained with A516, V468 or V26. Statistical analysis of the data in FIG. 8(a): **p<0.01 (comparison with scrapie-free equivalent).

### EXAMPLES

### Materials & Methods

### Sheep Inoculations

Scrapie-free VRQ homozygous Cheviot lambs were derived by embryo transfer from ewes of UK origin. Lambs were born and inoculated with scrapie within a purpose built experimental facility isolated from other livestock. Six lambs, at 1-2 weeks of age, were dosed orally with 1 g of undiluted scrapie-infected sheep brain homogenate placed on the back of the tongue.

The homogenate was prepared from brains of scrapie-infected homozygous or heterozygous VRQ and ARQ sheep. Inoculated lambs were maintained under normal sheep husbandry conditions and monitored for clinical signs of scrapie. Three animals were culled at 6 months post-scrapie inoculation and the three remaining animals were maintained until they displayed unequivocal signs of scrapie, at which point they were euthanazed. Scrapie infection was confirmed in all clinical cases by the presence of disease-specific PrP in brain sections and by typical vacuolar pathology in the brain stem.

### Monoclonal Antibodies

Anti-PrP monoclonal antibodies used here were produced as described previously (Thackray, A. M. et al. (2004) Biochem. J. 381 221-9). The N terminal-specific anti-PrP monoclonal antibodies T164, T188 and T325 were produced from Prnp^{∘/∘} mice immunized with murine brain homogenate. The C-terminal-specific anti-PrP monoclonal antibodies A516, V468 and V26 were produced from Prnp^{∘/∘} mice immunized with ovine recombinant PrP.

### Blood Sampling

Peripheral blood was collected by jugular venepuncture from scrapie-inoculated sheep, or from age- and sex-matched New Zealand-derived scrapie free sheep. Scrapie inoculated lambs were bled immediately prior to prion challenge and subsequently at monthly intervals post-scrapie inoculation. Blood was either collected in EDTA tubes or allowed to clot and was subsequently transported on ice and stored overnight at 4° C. prior to preparation of PBMCs.

Isolation of sheep PBMCs for immunofluorescence staining A buffy coat was prepared from EDTA-treated blood by centrifugation at 733 g for 20 min at 21° C. and the harvested cells were layered onto NycoPrep™ Animal (density 1.077 g/ml; osmolarity 265 mOsm), and centrifuged at 600 g for 15 min at 21° C. Mononuclear cells were recovered from the density medium interface and washed three times with FACS buffer (PBS containing 1% heat-inactivated foetal calf serum [FCS] supplemented with 0.1% sodium azide) prior to immunofluorescence staining. Cell surface phenotype was assessed using aliquots of 1×10⁶ cells incubated with monoclonal antibody culture supernatant, or normal mouse serum at 1:1000 as control, for 20 min at 4° C. followed by three washes in FACS buffer and incubation with goat anti-mouse IgG-biotin (Sigma, cat. no. B-7264) at 1:1000 or goat anti-mouse IgG1-biotin (Caltag, cat. no. M32115) or anti-mouse IgG2a-biotin (Caltag, cat. no. M32215) or anti-mouse IgM-biotin (Caltag, cat. no. M31515) all at 1:500, for 20 min at 4° C. Cells were washed three times with FACS buffer and subsequently incubated with 0.25 μg of streptavidin-phycoerythrin (Pharmingen, cat. no. 554061) for 20 min at 4° C. Cells were finally washed three times with FACS buffer and analyzed for cell surface fluorescence using a FACSCalibur (Becton Dickinson, Mount View, Calif., USA). Ten thousand cells were analyzed per sample with dead cells excluded on the basis of forward and side light scatter.

### Immunohistochemistry

The detection of PrP^{Sc} by immunohistochemistry was performed as previously described (Terry, L. A. et al (2003) Vet. Rec. 152, 387-92). Briefly, 5 μm sections from each of three wax blocks per anatomical site (brain and lymphoid tissue) from scrapie-infected sheep killed at serial time points were stained with R145, a C-terminal-specific rat anti-PrP monoclonal antibody raised against bovine PrP peptide sequence residues 221-232. Sections were dewaxed, endogenous peroxidase quenched and slides were treated with 98% formic acid for 5 min then autoclaved at 121° C. for 5 min in citrate buffer (pH 6). After blocking with normal rabbit or goat serum, the primary antibody was applied for 1 h at 21° C., followed by detection with biotinylated rabbit anti-rat or goat anti-mouse anti-serum (Vector Laboratories) and citrate buffered diaminobenzidine for visualization. The sections were counterstained with Mayer's hematoxylin and examined under bright-field microscopy.

### Statistical Analysis

Statistical analysis of the data was performed using the 2-tailed Students t-test (paired samples).

### Nomenclature

Amino acid residue numbers refer to the ovine PrP sequence.

### Results

### Efficient Development of Scrapie Disease In Orally Inoculated Homozygous VRQ Lambs

A novel experimental model of scrapie in homozygous VRQ lambs inoculated orally with scrapie-infected brain homogenate was employed. All inoculated lambs displayed classical signs of scrapie, with the earliest clinical signs seen at 3 months post inoculation (p.i.) when behavioural changes such as excitability and slight nervousness were evident.

Over the next 2 months the inoculated sheep displayed similar signs with intermittent nervousness and anxiety. Only in the last 2 months did the scrapie-infected sheep show signs of scratching and pruritus. These features are typical of scrapie disease and confirmed that all of the animals in this model system were susceptible to scrapie. The incubation period from scrapie inoculation to terminal scrapie disease was 216±12 days. A similar incubation time was recorded in a duplicate experiment using similar aged VRQ lambs inoculated with a similar dose of scrapie brain homogenate. This confirms that oral inoculation of young VRQ lambs leads to efficient development of scrapie infection that culminates in disease with a relatively short incubation period.

In a parallel study, homozygous VRQ lambs inoculated in a similar manner were sacrificed at various time points when peripheral lymphoid tissue and brain tissue was analyzed for the presence of PrP^{Sc} by immunohistochemistry. Inoculated lambs showed the presence of PrP^{Sc} in distal ileum Peyer's patches from as early as 21 days p.i. PrP^{Sc} was subsequently detected in other peripheral lymphoid sites, including mesenteric, prescapular and popliteal lymph nodes. Both TBMs (intense granular depositions) and FDCs (diffuse staining) were positive for PrP^{Sc} and similar patterns of staining were seen in other peripheral lymphoid tissue sites. PrP^{Sc} was detected in the brain from 110 days p.i. in the same animals.

### Increased Expression of N-Terminal PrP Epitopes By PBMCs From Scrapie-Infected Sheep

FIG. 2 shows the reactivity of PBMCs from scrapie-infected sheep with anti-PrP monoclonal antibodies that are reactive with the N-terminal region of the prion protein. T164, T188 and T325 all have specific epitopes within amino acid residues 25-88 of ovine PrP. Whilst these anti-PrP monoclonal antibodies recognize epitopes within the N-terminal region of ovine PrP, they showed variation in their reactivity with blood cells from scrapie-infected sheep. At 3 months, post-scrapie inoculation, T325 reacted with virtually all of the PBMCs from scrapie-infected sheep, whilst T188 and T164 reacted with less cells. T164 reacted with less than 40% of PBMCs at this time point. The absence of T164 and T188 reactivity with PBMCs from scrapie-infected sheep was not sustained throughout the course of the incubation period. At subsequent time points there was an increase in the number of T164 and T188 reactive PBMCs (FIG. 2). At 5 months p.i., and at later time points, the percentage of cells reactive with each of the N-terminal monoclonal antibodies approached that seen with T325, although the percentage of T164-reactive cells was always less than the percentage of T188 or T325 positive cells.

Representative flow cytometry profiles for T164, T188 and T325 reactive with PBMCs from scrapie-infected sheep are shown in FIG. 3. At 3 months p.i., monoclonal antibody T164 clearly reacted with less than 50% of the PBMCs from scrapie-infected sheep. At later time points, as the percentage of T164-positive cells increased, it became clear that there were T164-high and T164-low positive cells. T325 showed a uniform single reactivity profile with virtually all of the PBMCs binding this monoclonal antibody at all of the time points analyzed.

### Variation In C-Terminal PrP Epitope Accessibility On PBMCs From Scrapie-Infected Sheep

FIG. 4 shows the percentage of PBMCs from scrapie-infected sheep reactive with monoclonal antibodies A516, V26 and V468. These monoclonal antibodies were generated against truncated ovine PrP that lacked amino acid residues 25-88 and are therefore reactive in the C-terminal portion of the molecule. At all of the time points investigated, monoclonal antibody A516 showed reactivity with the majority of PBMCs from scrapie-infected sheep. In contrast, monoclonal antibodies V26 and V468 showed variation in their reactivity with PBMCs from scrapie-infected sheep. V26 did not show significant reactivity with PBMCs at the earliest time point measured. However, at subsequent time points there was a progressive increase in the percentage of cells reactive with this monoclonal antibody.

At 7 months p.i. approximately 50% of the PBMCs reacted with monoclonal antibody V26. In contrast, monoclonal antibody V468 showed a reciprocal reactivity to that seen with V26 during the course of the experiment. The percentage of V468-positive cells decreased from approximately 75% to 25% over the course of the experiment.

### PrP Expression By PBMCs From Age-Matched Scrapie-Free Or Scrapie-Infected Sheep

To exclude the possibility that the changes in PrP expression seen on the surface of blood cells isolated from scrapie-infected sheep were not simply an age-related phenomenon, N-terminal and C-terminal FACS staining of PBMCs from age-matched New Zealand-derived scrapie-free homozygous VRQ sheep was investigated.

FIGS. 5 and 6 show representative FACS plots of PBMCs from scrapie-free and scrapie-infected sheep stained with either N-terminal or C-terminal specific anti-PrP monoclonal antibodies. FIG. 5 shows that there were fewer PBMCs from scrapie-free control sheep reactive with the N-terminal monoclonal antibodies T164 and T188 compared with PBMCs from scrapie-infected animals.

T325 showed similar reactivity with PBMCs from both scrapie-free and scrapie-infected animals. FIG. 6 shows that the C-terminal monoclonal antibody V26 had little, if any, reactivity with PBMCs from scrapie-free sheep but showed significant reactivity with PBMCs from age-matched scrapie-infected sheep. Monoclonal antibodies A516 and V468 showed similar reactivity with PBMCs from both scrapie-free and scrapie-infected animals.

FIGS. 7 and 8 show the quantitation of the N-terminal and C-terminal FACS analysis of PBMCs from scrapie-free and scrapie-infected sheep. FIG. 7 (a) shows that the percentage of PBMCs from scrapie-infected sheep reactive with N-terminal monoclonal antibodies was significantly greater than that seen with PBMCs from scrapie-free sheep. PBMCs from scrapie-infected sheep showed a significantly higher percentage of cells reactive with T325 compared with age-matched scrapie-free controls (p<0.05). This trend was also seen when the PBMCs were stained with T188 (p<0.01) or T164 (p<0.001). In addition, the fluorescence intensity of all three N-terminal-specific monoclonal antibodies was significantly greater with PBMCs from scrapie-infected sheep compared with cells from scrapie-free animals as shown in FIG. 7 (b). PBMCs from scrapie-infected sheep gave a significantly higher mean channel number with T325 compared with age-matched scrapie free controls (p<0.001). This trend was also seen when the PBMCs were stained with T188 or T164 (p<0.01). FIG. 8 (a) shows that the percentage of PBMCs from scrapie-infected sheep reactive with monoclonal antibody V26 was significantly greater than that seen with PBMCs from scrapie-free sheep (p<0.01).

Our experiments have shown that PBMCs isolated from scrapie inoculated lambs show structural variation in PrP on their cell surface compared to similar cells from scrapie-free sheep.

Changes in the C-terminal region of PrP on PBMCs from scrapie-infected sheep were evident as seen by the differential reactivity of monoclonal antibodies specific for epitopes in the globular domain of the protein. Monoclonal antibody V26 reacted with a progressively increasing percentage of cells during the course of scrapie disease. In contrast, other monoclonal antibodies such as V468, which recognizes an epitope that includes, or is influenced by, residue 171, reacted with progressively fewer cells. Cell surface PrP with changes in the region around residue 171 was further indicated by the monoclonal antibodies 968 and 683, which react with the epitopes YYRPVD (amino acid residues 165-170) and PVDQY (amino acid residues 168-172) respectively. These monoclonal antibodies do not bind to cell surface PrP expressed by PBMCs from scrapie-free sheep but did show variable reactivity with cells from scrapie-infected sheep. The variation in reactivity of monoclonal antibodies V468, 683 and 968 is suggestive of changes in epitope exposure between the second β-strand and helix-2, a region of cell surface PrP which has been regarded as partially buried or obscured in normal PrP^{C}. The changes in epitope exposure by PrP expressed on the surface of ovine PBMCs during scrapie disease does not appear to be an age-related effect. Cells prepared from age-matched scrapie-free sheep did not show similar PrP expression profiles. In addition, the changes in PrP expression did not appear to be due to the appearance of a unique cell type in peripheral blood since there was no significant change in the scatter profiles of PBMCs during the progression of scrapie disease. Secondly, there was no significant difference in the numbers of PBMCs in the blood from scrapie-infected and scrapie-free sheep.

Our results are consistent with the appearance of altered conformers of PrP on the surface of blood cells from scrapie-infected sheep. Indirect evidence in support of this comes from Carmona et al (2004) who have shown through the use of Raman spectroscopy that a membrane preparation from scrapie-infected sheep blood cells has a significantly higher β-sheet content compared to normal cells. As PrP^{C} conversion to PrP^{Sc} involves an increase in β-sheet content, blood cell PrP is considered to be the candidate protein responsible for the increased β-sheet content within the membrane fraction.

1. A method of detecting prion disease in an individual comprising;
contacting blood cells obtained from said individual with an antibody which specifically binds to PrP^{c}, and
determining the binding of said antibody to said cells.

2. A method according to claim 1 wherein a change in the binding of said antibody relative to controls is indicative of the presence of prion disease in the individual.

3. A method according to claim 1 wherein said antibody specifically binds to residues 25 to 88 of ovine PrP.

4. A method according to claim 3 wherein the antibody is selected from the group consisting of T188, T164 and T325

5. A method according to claim 1 wherein said antibody specifically binds to residues 89 to 233 of ovine PrP.

6. A method according to claim 5 wherein said antibody specifically binds to residues 217 to 232 of ovine PrP.

7. A method according to claim 6 wherein the antibody is a V26 antibody.

8. A method according to claim 7 wherein the antibody specifically binds to the epitope CITQYQRESQAYYQRG (SEQ ID NO:1).

9. A method according to claim 5 wherein said antibody specifically binds to an epitope comprising or influenced by residue 171 of ovine PrP.

10. A method according to claim 9 wherein the antibody is a V468 antibody.

11. A method of detecting prion disease in an individual comprising;
contacting blood cells obtained from said individual with an antibody which specifically binds to the epitope CITQYQRESQAYYQRG (SEQ ID NO:1), and
determining the binding of said antibody to said cells
wherein increased binding of said antibody to said cells relative to controls is indicative of the presence of prion disease in the individual.

12. A method according to claim 1 wherein the blood cells are contacted with two or more antibodies which specifically bind to PrP, and the binding of the two or more antibodies to said cells determined.

13. A method according to claim 1 wherein the blood cells are peripheral blood mononuclear cells (PBMCs).

14. A method according to claim 13 wherein the PBMCs are isolated from a blood sample obtained from the individual.

15. A method according to claim 14 wherein the PBMCs are isolated by density gradient centrifugation.

16. A method according to claim 1 wherein binding of said antibody to said cells is determined by flow cytometry.

17. A method according to claim 1 wherein the individual is a non-human animal.

18. A method according to claim 17 wherein the prion disease is CJD, BSE, scrapie or chronic wasting disease.

19. A kit for detecting prion disease or assessing the progression of a prion disease in an individual comprising:
one or more antibodies which specifically bind to PrP, and;
detection reagents for determining binding of said antibody to blood cells.

20. A kit according to claim 19 wherein said one or more antibodies comprise an antibody which specifically binds to residues 25 to 88 of ovine PrP.

21. A kit according to claim 20 wherein the antibody is selected from the group consisting of T188, T164 and T325.

22. A kit according to claim 19 wherein said one or more antibodies comprise an antibody which specifically binds to residues 89 to 233 of ovine PrP.

23. A kit according to claim 22 wherein said antibody specifically binds to residues 217-232 of ovine PrP.

24. A kit according to claim 23 wherein said antibody is a V26 antibody.

25. A kit according to claim 23 or claim 24 wherein said antibody specifically binds to the epitope CITQYQRESQAYYQRG (SEQ ID NO:1).

26. A kit according to claim 22 wherein said antibody specifically binds to an epitope comprising, or influenced by, residue 171 of ovine PrP.

27. A kit according to claim 26 wherein the antibody is a V468 antibody.

28. A kit according to claim 19 comprising apparatus for handling and/or storing blood cells.

29. A kit according to claim 19 comprising reagents for isolating and/or purifying blood cells from a blood sample.

30. A kit according to claim 19 wherein the blood cells are peripheral blood mononuclear cells (PBMCs).

31. A kit according to claim 19 wherein the detection reagents comprise a second antibody which binds to the anti-PrP antibody.

32. A kit according to claim 19 wherein the anti-PrP antibody or the second antibody are labelled with a detectable reporter.

33. A kit according to claim 19 wherein the anti-PrP antibody or the second antibody are labelled with a tag which binds a detectable reporter.

34. A kit according to claim 33 wherein the detection reagents comprise a detectable reporter that binds to the tag.

35. A kit according to claim 19 wherein the individual is a non-human animal.

36. A kit according to claim 35 wherein the prion disease is chronic wasting disease, BSE or scrapie.

37. An antibody which specifically binds to the epitope CITQYQRESQAYYQRG (SEQ ID NO:1).

38. An antibody according to claim 37 which is labelled with a fluorescent label.

39. A hybridoma cell which produces an antibody according to claim 37.

40. A method of producing an antibody comprising;
culturing a hybridoma cell according to claim 39 in a culture medium; and,
isolating said antibody from said medium.

41. A method of producing a hybridoma according to claim 39 comprising;
immunising a non-human mammal with a peptide consisting of the sequence CITQYQRESQAYYQRG (SEQ ID NO:1),
producing one or more fusions of antibody producing cells from said mammal and immortalised cells to provide a population of hybridoma cells and;
screening said population to identify a hybridoma cell which produces an antibody which binds to said peptide.

42. A method according to claim 41 comprising isolating and/or purifying said hybridoma from said population.

43. A method according to claim 42 comprising maintaining and/or culturing said hybridoma in a culture medium.

44. A method according to claim 43 comprising isolating an antibody which binds said peptide from said culture medium.
